(19) United States
(12) Plant Patent Application Publication
(10) Pub. No.: US 2002/0194658 P1
(43) Pub. Date: Dec. 19, 2002

(54) MAURANDYA PLANT NAMED 'MAGIC DRAGON'

(76) Inventor: Ray Brown, Newton Abbott (GB)

Correspondence Address: MARK P. BOURGEOIS, P.O. BOX 95, OSCEOLA, IN, 46561, US

(21) Appl. No.: 09/883,420

(22) Filed: Jun. 18, 2001

(51) Int. Cl. A01H 5/00

(52) U.S. Cl. PLT/226

(57) ABSTRACT

A new cultivar of Maurandya named ‘Magic Dragon’ that is characterized by semi-weeping branches and light green foliage, large bright red trumpet-shaped flowers with flared lobes, and a climbing habit when secured to a stake or trellis. In combination these traits set ‘Magic Dragon’ apart from all other existing varieties of Maurandya.

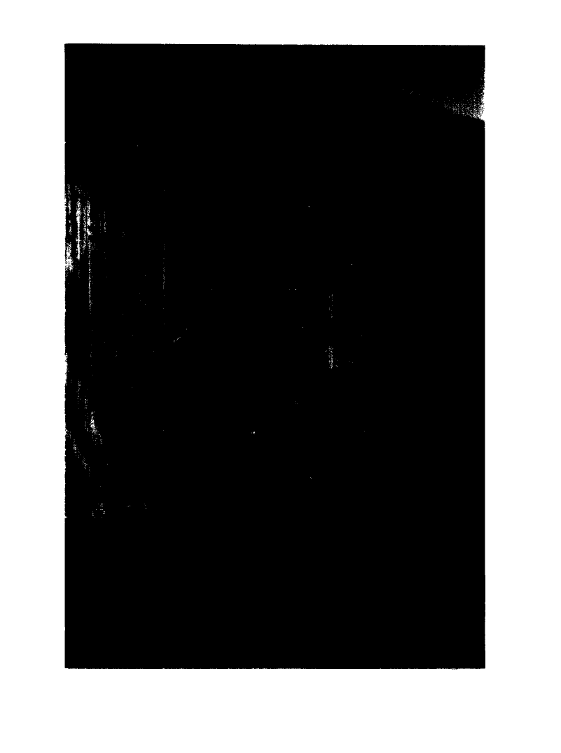

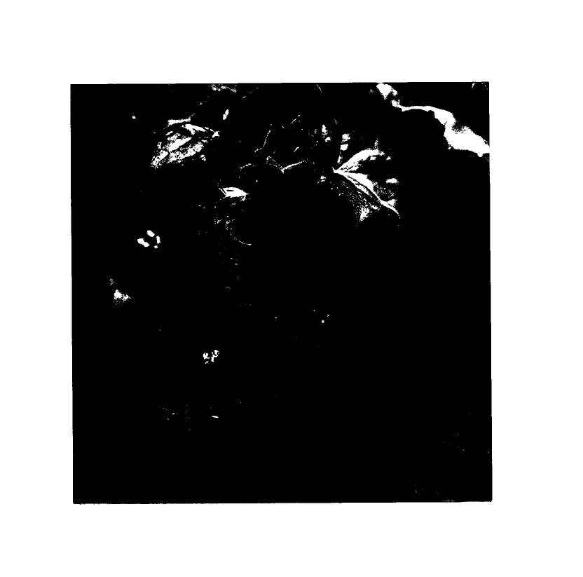

### BACKGROUND OF THE INVENTION

The present invention relates to a new and distinct cultivar of Twining Snapdragon known botanically as Maurandya and referred to hereinafter by the cultivar name ‘Magic Dragon’. The present invention was selected by the inventor in July 1995 in Newton Abbot, United Kingdom. The new invention is the product of the inventors breeding program. The purpose of the program was to produce a plant that will exhibit a climbing habit, while producing the bright red flowers of ‘Red Dragon’ and the larger flared trumpet flowers of Maurandya erubescens.

‘Magic Dragon’ is a hybrid that resulted from the induced hybridization of the female parent Maurandya ‘Red Dragon’ (not patented) and the male parent Maurandya erubescens (riot patented). Correct nomenclature listed in The Royal Horticultural Society Dictionary of Gardening for Maurandya, ‘Red Dragons’ includes the synonym Asarina ‘Red Dragon’ and correct nomenclature for Maurandya erubescens includes the synonym Asarina lophospermum.

‘Magic Dragon’ is distinguished by light green foliage, increased vigor over the parent plants, a climbing habit when secured to a stake or trellis, and large blight red, trumpet-shaped flowers with flared lobes. The closest comparison plants are the parents. ‘Magic Dragon’ differs from ‘Red Dragon’ in its climbing habit when secured and larger flared trumpet flowers. ‘Magic Dragon’ is distinguishable from Maurandya erubescens by the bright red color of its flowers.

‘Magic Dragon’ was first asexually propagated by the inventor in Newton Abbot, England in June 1996. Asexual propagation was accomplished using vegetative tip cuttings. Since that time, under careful observation, successive generations have been determined fixed, stable and uniform.

### SUMMARY OF THE INVENTION

The following represent the distinguishing characteristics of the new Maurandya cultivar ‘Magic Dragon’. In combination these traits set ‘Magic Dragon’ apart from all other existing varieties of Maurandya. ‘Magic Dragon’ has not been tested under all possible conditions and phenotypic differences may be observed with variations in environmental, climatic, and cultural conditions, however, without any variance in genotype.

1. ‘Magic Dragon’ exhibits semi-weeping branches and light green foliage.

2. Maurandya ‘Magic Dragon’ exhibits large, flared. bright red trumpet-shaped flowers.

3. Maurandya ‘Magic Dragon’ exhibits a climbing habit when secured to a stake or trellis.

4. Maurandya ‘Magic Dragon’ is 2 meters in height at maturity.

5. Maurandya ‘Magic Dragon’ is hardy to USDA Zone 9.

6. Maurandya ‘Magic Dragon’ blooms in spring and summer,

### BRIEF DESCRIPTION OF THE DRAWINGS

The accompanying color photographs illustrate the distinguishing traits of ‘Magic Dragon’. The plants in the photographs are ten months old and were grown outside in fifteen-gallon containers. The photograph on sheet 1 illustrates an entire specimen of ‘Magic Dragon’ in bloom from a side perspective. The photograph on sheet 2 is a close-up photograph illustrating the flower and leaf. All photographs are taken using conventional techniques and although foliage colors may appear different from actual colors due to light reflectance, they are as accurate as possible by conventional photography.

### BOTANICAL DESCRIPTION OF THE PLANT

The following is a detailed description of the Maurandya cultivar named ‘Magic Dragon’. Data was collected in Arroyo Grande, Calif. from ten month old plants grown outside in five-gallon containers. Color determinations are in accordance with the Royal Horticultural Society Colour Chart except where general color terms of ordinary dictionary significance are used. The growing requirements are similar to the species and no disease problems have been observed.

Botanical classification: Maurandya ‘Magic Dragon’

Common name: Twining snapdragon.

Use: Ornamental container vine or trellis.

Parentage: Maurandya ‘Magic Dragon’ is the result of an induced hybridization between the following plants:

Female parent.—Maurandya ‘Red Dragon’.

Male parent.—Maurandya erubescens . . .

Growth rate.—Vigorous to aggressive.

Growth habit.—Scandent when secured to a stake or trellis.

Height.—2 meters or more in height at maturity.

Width.—40-60 cm. in width.

Hardiness.—USDA Zone 9.

Propagation.—Propagated by vegetative tip cuttings.

Root system.—Fibrous.

Cultural requirements.—Plant in well-drained soils and full sunlight. Type: Perennial.

Time to initiate roots.—2-3 weeks are required at 25° Centigrade for cuttings to root.

Time to develop to a five-gallon container.—3-4 months is required to develop a finished five-gallon container from a rooted cutting.

Branching habit.—Semi-weeping.

Seasonal interest.—Large blight red flowers in spring and summer.

Stem.—Stem shape: Cylindrical Dimensions of stem: 0.25 cm. wide and 35 cm. in length Internode length: 1.5 to 3 cm. between nodes Stem diameter: 2.5 cm. in diameter Stem length: 10.5 cm. in length Surface: Tomenitose and glandular with beads of sticky fluid Color of tomenta: 197B Color of stem: 146C.

Foliage.—. Type: Evergreen Leaf arrangement: Alternate Leaf division: Simple Leaf shape: Deltoid Leaf base: Sagitate Leaf tip: Aristulate Leaf venation: Palmat Leaf attachment: Petiolate Petiole dimensions: 2 mm in width and 1.5-2 cm. in length Petiole surface: Heavily pubescent Color of petiole: 199B Leaf margin: Biserrate Leaf length: 4 cm in length Leaf width: 3.5-4 cm in width Quantity of leaves: Approximately 17 leaves per stem Leaf color (adaxial and abaxial surface): 144A Foliar fragrance: None observed

Flower.—Form: Solitary Flower arrangement: Axillary Shape of flower: Tubular to trumpet with flared lobes and exhibiting a tumid region on the ventral basal surface of the corolla tube Flower symmetry: Irregular and bilabiate Sexuality: Bisexual Dimensions of nectary: 2.5 cm. in length and 1-2 cm. in width Color of nectary: 15A Surface of nectary: Bearded Region of nectary: Two rows running lengthwise along the ventral basal surface of the corolla tube Flower aspect: Facing outward Color of flower petal: 185A with tint of 64B on the lobes Petals: Five in number Petal surface: Smooth surface Dimensions of corolla tube: 5 cm. in length and 3 cm. in diameter Corolla lobes: Five in number Dimensions of lobes: Each lobe is 1.5 cm. in width and 1 cm. in length Color of Corolla tube: 185A Petals unfused or fused: Sympetalous Bud color: 185A Bud dimensions: 3 cm. in length and 1 cm. in width Persistent or self-cleaning: Self-cleaning Shape of Calyx: Tubular when closed and stellate when open Color of calyx: 144A. Dimensions of calyx: 3 cm. in length and 2.5 cm. in diameter Number of sepals: Five sepals basally united Sepal color: 144A Blooming period: Spring and summer Pedunclc dimensions: 2 mm. in width and 3.5 cm. in length Color of peduincle: 199A Surface of peduncle: Stipitate glandular surface Fragrance: None observed.

Reproduction organs.—Stamens: Four in number that rest against the ventral surface of the corolla tube opposite the nectary. Form of stamen: Included. Color of filament: 155B suffused with 59C at base Dimensions of stamen: 2.5-3 cm. in length and 1 mm in diameter Arrangement of stamens: Didynamous Anther type: Adhering Anther color: 155B Dimensions of anther: 3 mm. in width and 3 mm. in length Pollen color: 158D Amount of pollen: moderate Pistil: One in number Pistil color: 157A. Dimensions of pistil: 4.5-5 cm. in length and less than .50 mm. in diameter Form of stigma: Stigmas are united Stigma color: 161A Dimensions of stigma: 1 mm. in height and 1.25 mm. in diameter Style color: 155D Style dimensions: 1 mm. in length and less than .50 mm. in diameter Ovary position: Superior Ovary color: 145CB Ovary dimensions: 2 mm. in diameter and 1 mm. in height Ovary shape: Globose

Seed.—Seed production has not been observed.

1. A new and distinct variety of Maurandya plant named ‘Magic Dragon’ as illustrated and described:
